(19) United States
(12) Patent Application Publication
(10) Pub. No.: US 2024/0177840 A1
(43) Pub. Date: May 30, 2024

(54) SERVICE APPARATUS FOR IMPROVING DAILY BEHAVIORS OF PEOPLE WITH DEVELOPMENTAL DISORDERS AND METHOD OF OPERATING THE SAME

(71) Applicant: eMOTIV Co., Ltd., Seoul (KR)

(72) Inventors: Jung Sang MIN, Seoul (KR); Ga Young KIM, Seoul (KR); Sung Jin KIM, Seoul (KR); Sol Yeon JEON, Seoul (KR); Min Jung JI, Seoul (KR)

(21) Appl. No.: 18/511,934

(22) Filed: Nov. 16, 2023

(30) Foreign Application Priority Data
Nov. 29, 2022 (KR) 10-2022-0162764

(51) Int. Cl. G16H 40/00; G16H 10/60

(52) U.S. Cl. CPC G16H 40/00; G16H 10/60

(57) ABSTRACT

A service apparatus according to an embodiment of the present disclosure may include a user account management unit that manages accounts of a person with developmental disorder and a guardian of the person with developmental disorder; a training service information output unit that outputs training service information for improving the daily behavior of the person with developmental disorder; and a mode switching service unit that processes mode switching between a guardian mode and a person-with-developmental-disorder mode to output the training service information.

### BACKGROUND OF THE INVENTION

### Cross-Reference to Related Applications

This application claims priority under 35 U.S.C § 119 to Korean Patent Application No. 10-2022-0162764 filed in the Korean Intellectual Property Office on Nov. 29, 2022, the entire contents of which are hereby incorporated by reference.

### Field of the Invention

The present disclosure relates to relates to a service apparatus and a method of operating the same. More specifically, the present disclosure provides training services and game services on a method of improving daily behaviors for people suffering from developmental disorders such as autism spectrum disorders, and relates to a service apparatus for improving daily behaviors of people with developmental disorders to aid children in facilitating independent daily lives, and a method of operating the same.

### BACKGROUND OF THE RELATED ART

Developmental disorders do not refer to any specific diseases or disabilities, but refer to all conditions characterized by delays and abnormalities in social relationships, communication, and cognitive development, and refer to any conditions in which a person does not develop appropriately for his or her age.

Furthermore, in developmental disorders, the growth rate of language, cognition, movement, and social skills may be significantly slower than that of peers, resulting in reduced self-help skills that can be used in real life, slow language acquisition and motor development, and symptoms of poor interaction.

Such developmental disorders are disorders that mainly occur in childhood, and cerebral palsy caused by brain lesions during the period of birth when the brain is immature, intellectual disability, which is a condition in which learning is impaired due to impaired intellectual development, and autism or autism spectrum disorder (ASD), which is characterized by impaired social interaction, verbal and non-verbal communication, stereotyped behaviors, and interests in childhood are included in related diseases thereof.

In particular, among developmental disorders, autism spectrum disorder (ASD) has poor or infrequent eye contact, facial expressions, and gesture use, and is unable to form peer relationships appropriate for developmental level. Additionally, delayed oral language development and limited, repetitive, and stereotyped behaviors and interests are the main symptoms of autism spectrum disorder.

Accordingly, the sooner treatment for patients with autism spectrum disorders (ASDs) is performed, the more effective the treatment will be, but if the treatment period is missed, it takes a long period of time, and there is a significant cost burden.

In addition, intensive treatment for improving the symptoms of autism spectrum disorder includes various treatments such as applied behavior analysis (ABA) treatment, which is known to be highly effective in improving symptoms, as well as drug treatment, special treatment, speech therapy, and psychological and social intervention.

Here, in order to quickly improve symptoms, when a patient undergoes other special treatments in addition to the ABA (Applied Behavior Analysis) treatment, the financial burden may increase due to significant medical expenses.

Moreover, for the treatment of children with autism spectrum disorders (ASDs), there are several applications that anyone can easily access, but most of the applications focus on improving children's social and social communication skills.

Besides, an application that help improve activities such as toileting and eating, which are prerequisites for social communication in children suffering from autism spectrum disorders (ASDs) has unfortunately not existed in Korea up to the present.

Likewise, since there is no application in Korea for the treatment of patients with developmental disorders, including autism spectrum disorders, parents are spending a lot of money to use specialized treatment programs for their children with developmental disorders, but those programs are not only expensive but also require treatment over a considerably long period of time, and thus there is an urgent need for a more convenient, cheaper, and more efficient treatment method to improve developmental disorders.

### SUMMARY OF THE INVENTION

The present disclosure is contrived to solve the problems described above, and an aspect of the present disclosure is to perform a training service for improving daily behaviors of people with developmental disorders such as autism spectrum disorders, provide a training character reflecting the training service, and provide pre-set reward data to those with developmental disorders as a reward system for the training service, thereby providing a service apparatus for improving the daily behaviors of those with developmental disorders capable of providing a simple and inexpensive training process without the intervention of an attending physician or therapist so as to increase a training immersion level of those with developmental disorders and a method of operating the same.

In order to solve the foregoing problems, a service apparatus according to an embodiment of the present disclosure may include a user account management unit that manages accounts of a person with developmental disorder and a guardian of the person with developmental disorder; a training service information output unit that outputs training service information for improving the daily behavior of the person with developmental disorder; and a mode switching service unit that processes mode switching between a guardian mode and a person-with-developmental-disorder mode to output the training service information.

In addition, in order to solve the foregoing problems, a method of operating a service apparatus according to an embodiment of the present disclosure may include managing accounts of a person with developmental disorder and a guardian of the person with developmental disorder; outputting training service information for improving the daily behavior of the person with developmental disorder; and processing mode switching between a guardian mode and a person-with-developmental-disorder mode to output the training service information.

According to an embodiment of the present disclosure, a training service may be carried out to improve daily behaviors of people with developmental disorders, a training character reflecting the training service may be provided, and pre-set reward data may be provided to those with developmental disorders as a reward system for the training service, thereby having an effect of encouraging those with developmental disorders to follow improved daily behaviors using their preferred characters as well as performing highly immersive training services.

In addition, guardians of people with developmental disorders may be allowed to use a service apparatus to more easily set a training process for training services with various detailed items in a direct manner so as to provide not only training services in various approaches but also various game services to those with developmental disorders, thereby providing a service apparatus for improving daily behaviors of those with developmental disorders capable of reducing the time and financial costs of the guardians of those with developmental disorders as a treatment program with less time constraints and a method of operating the same.

### BRIEF DESCRIPTION OF THE DRAWINGS

FIG. 1 is a diagram showing in more detail a service apparatus according to an embodiment of silver present disclosure.

FIGS. 2A, 2B, 2C and 2D are diagrams showing an example of training services provided by a service apparatus according to an embodiment of the present disclosure.

FIGS. 3A and 3B are diagrams showing evaluations of training services in a service apparatus according to an embodiment of the present disclosure.

FIG. 4 is a diagram showing an example of a reward system for training services according to an embodiment of the present disclosure.

FIG. 5 is a diagram showing game services provided by a service apparatus according to an embodiment of the present disclosure.

FIGS. 6A and 6B are diagrams showing a reward system for game services according to an embodiment of the present disclosure.

FIG. 7 is a flowchart for explaining a service apparatus according to an embodiment of silver present disclosure.

### DESCRIPTION OF SYMBOLS

100: Service apparatus
110: User account management unit
120: Mode switching service unit
121: Guardian mode service unit
123: Person-with-developmental-disorder mode service unit
125: Database unit
130: Training service information output unit

### DETAILED DESCRIPTION OF THE PREFERRED EMBODIMENT

The following description illustrates the principles of the present disclosure. It will thus be appreciated that those skilled in the art will be able to devise various devices that, although not explicitly described or shown in this specification, implement the principles of the present disclosure and are included in the concept and scope of the present disclosure. Furthermore, it should be understood that all conditional terms and embodiments recited in this specification are intended only for pedagogical purposes to aid the reader in understanding the concept of the present disclosure, and are not limited to such specifically recited embodiments and conditions.

Moreover, it should be understood that all detailed description herein reciting the principles, aspects, and embodiments of the present disclosure as well as specific embodiments thereof are intended to encompass both structural and functional equivalents thereof. Additionally, it should be understood that such equivalents include both currently known equivalents as well as equivalents to be developed in the future, that is, any elements developed to perform the same function regardless of its structure.

Thus, for example, it should be understood that the block diagrams presented herein represent the conceptual view of an exemplary circuit that embodies the principles of the present disclosure. Similarly, it should be understood that any flow charts, flow diagrams, state transition diagrams, pseudocodes, and the like represent various processes which may be substantially represented in computer readable media and so executed by a computer or processor, whether or not such a computer or processor is explicitly shown.

The functions of various elements including a processor or a functional block represented as a concept similar thereto and shown in the accompanying drawings may be provided using hardware having a capability to execute appropriate software as well as dedicated hardware. When provided by the processor, the functions may be provided by a single dedicated processor, a single shared processor, or a plurality of individual processors, and some thereof may be shared with one another.

In addition, it should be understood that explicit use of the terms presented as processors, controls, or concepts similar thereto should not be interpreted by exclusively quoting hardware having an ability of executing software, and should be understood to implicitly include, without limitation, digital signal processor (DSP) hardware, and a ROM, a RAM and a non-volatile memory for storing software. Other known common hardware may also be included.

In the claims of this specification, an element expressed as a means for performing a function described in the detailed description is intended to include, for example, any method of performing a function including a combination of circuit elements or any form of software including firmware/microcode, and the like, which perform the function, and is combined with suitable circuitry for executing the software to perform the function. It should be understood that since functions provided by the various recited means are combined with one another and are combined with a scheme demanded by the claims in the present disclosure defined by the claims, any means capable of providing these functions are equivalent to means recognized from this specification.

The foregoing objects, features and advantages will be more obvious through the following detailed description associated with the accompanying drawings, and accordingly, the technological concept of the present disclosure can be easily implemented by a person having ordinary skill in the art to which the present disclosure pertains. In describing the present disclosure, moreover, the detailed description will be omitted when a specific description for publicly known technologies to which the present disclosure pertains is judged to obscure the gist of the present disclosure.

Hereinafter, a preferred embodiment according to the present disclosure will be described in detail with reference to the accompanying drawings.

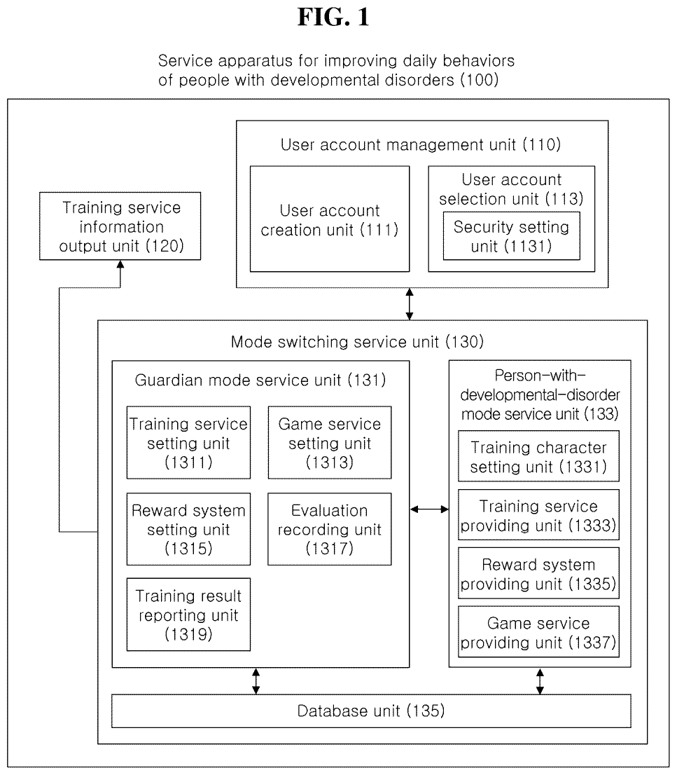

FIG. 1 is a diagram showing in more detail a service apparatus according to an embodiment of silver present disclosure.

Referring to FIG. 1, the service apparatus 100 according to an embodiment of the present disclosure, which is a service apparatus for improving the daily behavior of a person with developmental disorder, may be implemented as a server connected to a guardian terminal having a separate output apparatus through a wired/wireless network or implemented as application software installed on a guardian terminal itself.

Here, when the service apparatus 100 is implemented as a server connected to the guardian terminal, each of the networks may be implemented in all types of wired/wireless networks such as a Local Area Network (LAN), a Wide Area Network (WAN), a Value Added Network (VAN), a Personal Area Network (PAN), a mobile radiocommunication network, a satellite communication network, or the like.

Furthermore, when the service apparatus 100 is implemented as application software installed on the guardian terminal itself, it may be implemented as any one individual instrument of a computer, a mobile phone, a smart phone, a smart pad, a laptop computer, personal digital assistants (PDA), and a portable media player (PMP) capable of allowing a user input and a display output, or a kiosk installed in a specific location or at least one device of commonly-used devices such as a stationary display apparatus.

In addition, the service apparatus 100 may output training services and game services for improving the daily life of a person with developmental disorder, and may allow the guardian to directly record behavior data of the person with developmental disorder collected through the training services and game services into the service apparatus 100 so as to store and manage the results.

Additionally, the service apparatus 100 may monitor evaluation results for improvement in daily lives in real time based on recorded information on the behavior data. Moreover, the service apparatus 100 may continuously set up a reward system for the training services and game services in order to provide an effect of improving training motivation for the person with developmental disorder.

The training service data corresponding to the training service may be set differently depending on the type of training service, but an evaluation step of the training service may be preferably divided into several items according to the training service data in order to maximize the immersion and training effect for children suffering from major developmental disorders including autism spectrum disorders (ASDs).

More specifically, due to the nature of autism spectrum disorders (ASDs), the type and severity of problematic daily behavior may be different for each child, and the guardians of people with developmental disorders may divide an evaluation step in detail based on pre-configured training service data to provide appropriate training services to those with developmental disorders, and reflect the evaluation step in the training services to maximize an effect of improving lifelong behaviors for those with developmental disorders.

For this processing, the service device 100 includes a user account management unit 110, a training service information output unit 120, and a mode switching service unit 130.

First, the user account management unit 110 includes a user account creation unit 111 and a user account selection unit 113.

Here, the user account management unit 110 may manage accounts of a person with developmental disorder and a guardian of the person with developmental disorder.

More specifically, in the user account management unit 110, the personal information of a guardian of a person with developmental disorder and basic growth data including the name, gender, and date of birth of the person with developmental disorder are entered into the service apparatus 100 by the guardian so as to manage the accounts of the person with developmental disorder and the guardian.

Accordingly, the user account creation unit 111 may create a person-with-developmental-disorder account and a guardian account, respectively, for the person with developmental disorder and the guardian who uses the service apparatus to improve daily behavior in the service apparatus 100 according to an embodiment of the present disclosure.

Furthermore, the user account creation unit 111 may create a plurality of persons with developmental disorder when the guardian has two or more children with developmental disorder.

Additionally, the user account selection unit 113 may switch and select a type of user account according to a user mode switching input to the service apparatus 100.

Here, the user account selection unit 113 may include a security setting unit 1131, and the security setting unit 1131 may pre-set security input information to prevent the person with developmental disorder from accessing the guardian account when switching from the person-with-developmental-disorder account to the guardian account.

For example, the security setting unit 1131 may allow the guardian to set a password in advance on the service apparatus 100 or set a correct answer to a problem solving a simple calculation as security input information.

In addition, the training service information output unit 120 may output training service information for improving the daily behavior of the person of developmental disorder, and the output of the training service information may be configured in various forms. This training service information may be output directly from the service apparatus 100 or from a guardian terminal connected to the service apparatus 100 through a network. For this output, the training service information output unit 120, although not shown, may include a communication module, a display unit, and a voice output unit.

Additionally, the mode switching service unit 130 may process mode switching between a guardian mode and a person-with-developmental-disorder mode to output the training service information. Furthermore, for this mode switching, it includes a guardian mode service unit 131, a person-with-developmental-disorder mode service unit 133, and a database unit 135.

Here, the guardian mode service unit 131 may allow the guardian of the person with developmental disorder to set service data corresponding to the training service information, and may pre-set service data for processing when switching modes according to each service data. The service data may include training service data, game service data, reward data, and evaluation data.

More specifically, the guardian mode service unit 131 includes a training service setting unit 1311, a game service setting unit 1313, a reward system setting unit 1315, an evaluation recording unit 1317, and a training result reporting unit 1319.

Here, the training service setting unit 1311 may set training service data that sets a training service performed by the person with developmental disorder according to a user input of the guardian.

In addition, the training service setting unit 1311 may set a regular alarm function for an execution time of the training service to the training service data.

For example, the training service data may include information on preparations required for the training service selected by the guardian, information on an ongoing progress and method of the training service, information on precautions for performing the training service, information on a method of creating a training environment, and information on training goals, and may further include information on an evaluation guideline for the training service.

The training goal of the training service corresponding to the training service data may be customized to the person with developmental disorder according to the guardian's setting input to establish a customized training service.

In addition, the training service data may further include autism spectrum disorder (ASD) information, information on a list of problematic behaviors of people with developmental disorders, and information on training methods to improve symptoms of those with developmental disorders, which may be processed into a format such as columns, blog posts, and card news, and output in the form of a guide.

Furthermore, the game service setting unit 1313 may set the game service data for the game service performed by the person with developmental disorder.

Here, the game service may be preferably provided in a cooperative game format in which the person with developmental disorder and the guardian play in a cooperative manner, and a mini-game format in which the person with developmental disorder plays alone.

For example, the cooperative game may include a multi-player game in which the person with developmental disorder and the guardian play together to improve a lack of social skills that are the main symptoms of autism spectrum disorder (ASD) and a difficulty in forming bonds with others due to the lack of social skills to enhance sociality and connectedness.

In addition, the mini-game, which is a single-player game given to the person with developmental disorder along with the training service, may be a game played for a short period of time preferred by the person with developmental disorder, which may be composed of mini-games having an effect of enhancing the training motivation of the person with developmental disorder as well as improving his or her problematic daily behavior.

Furthermore, the game service setting unit 1313 may operate the background sound and effect sound control functions of the game service and the screen brightness control function of the user terminal, in consideration of the person with developmental disorder who is usually sensitive to sounds and colors, thereby setting the output sound and color to variables suitable for the person with developmental disorder.

In addition, the reward system setting unit 1315 may pre-set reward data for the guardian to provide the reward data for the training service and game service to the person with developmental disorder. Examples of the reward data may include stickers, and the like.

Here, the reward data may include stickers that can motivate the person with developmental disorder in performing the training service, and the reward system setting unit 1315 may process, when a certain amount of stickers are collected, condition information under which a final reward pre-set by the guardian is granted to the person with developmental disorder.

Furthermore, when the person with developmental disorder achieves 100% of the collected amount of stickers received as rewards that have been pre-set in the reward system setting unit 1315, a final reward pre-set by the guardian through the reward system setting unit 1315 may be granted to the person with developmental disorder. The reward system setting unit 1315 may set reward data to upload and record a photo of the final reward.

In addition, though the person with developmental disorder has already received a reward, the reward system setting unit 1315 may be provided with a daily additional reward process of providing additional reward data when clicking on a reward icon disposed in a certain region displayed through the guardian's user terminal.

Furthermore, the evaluation recording unit 1317 may record behavior data collected when the guardian evaluates the training service performed by the person with developmental disorder.

Besides, the training result reporting unit 1319 may calculate evaluation data based on the behavior data recorded in the evaluation recording unit 1317, and report the evaluation data as result data of the training service performed by the person with developmental disorder.

In addition, the evaluation recording unit 1317 may record reporting information of the result data, and the recorded reporting information may be stored and managed in the database unit 135 in real time.

The database unit 135 may be connected with the guardian mode service unit 131 and the person-with-developmental-disorder mode service unit 133, and even when mode switching is processed, information shared between each mode is equally stored, updated and managed.

Accordingly, the training result reporting unit 1319 may process a progress of the training service of the person with developmental disorder into a graph format using the evaluation data stored in real time in the database unit 135 according to the operation of the guardian mode service unit 131 and the person-with-developmental-disorder mode service unit 133, and output the processed graph through the training service information output unit 120, and also provide resultant feedback information through the training service information output unit 120.

For example, the training result reporting unit 1319 may output feedback information on the progress of the training service, such as ‘A success rate of 100% has been achieved in the last week’, through the training service information output unit 120.

Meanwhile, when the person with developmental disorder performs the training service, the guardian must record and store the behavior data in the service apparatus 100 in real time, and thus an operation mode may be preferably switched between the person-with-developmental-disorder mode service unit 133 and the guardian mode service unit 131. Accordingly, as described above, the database unit 135 may store the service data in real time to prevent loss of the service data when mode switching is processed by the mode switching service unit.

In addition, the person-with-developmental-disorder mode service unit 133 may be switched from the guardian mode service unit 131 based on operation mode switching according to a user input.

This person-with-developmental-disorder mode service unit 133 includes a training character setting unit 1331, a training service providing unit 1333, a reward system providing unit 1335, and a game service providing unit 1337.

Here, the training character setting unit 1331 may be set by the guardian mode service unit 131 to set a training character that reflects the training service based on the training service data stored in the database unit 135.

Additionally, the training service providing unit 1333 may output video content reflecting the training service in the set training character to allow the person with developmental disorder to easily perform the training service.

For example, the training service providing unit 1333 may output training service video content based on the training service data that reflects the training character settings, thereby allowing the person with developmental disorder to focus on performing the training service with enhanced immersion.

Furthermore, the training service may include, for example, a toilet training process, an eating training process, and a clothes change training process, and may output training service video content based on the training service data that reflects the training character settings, thereby visualizing each process of the training service, and guiding the person with developmental disorder to follow the training service.

In addition, the reward system providing unit 1335 may process the provision of reward data preset by the guardian when the guardian enters training service performance information as the person with developmental disorder performs the training service.

Here, the reward data may include stickers that can motivate the person with developmental disorder in performing the training service, and for an example, a final reward pre-set by the guardian may be granted to a character of the person with developmental disorder when a certain amount of stickers are collected.

Furthermore, the game service providing unit 1337 may provide the game service based on the game service data stored in the database unit 135 to the person with developmental disorder.

Here, the game service may be provided in a cooperative game format in which the person with developmental disorder and the guardian play in a cooperative manner, and a mini-game format in which the person with developmental disorder plays alone.

For example, the cooperative game may be a multi-player game in which the person with developmental disorder and the guardian play together to improve a lack of social skills that are the main symptoms of autism spectrum disorder (ASD) and a difficulty in forming bonds with others due to the lack of social skills to enhance sociality and connectedness.

In addition, the mini-game, which is a single-player game given to the person with developmental disorder along with the training service, and may be a game played for a short period of time preferred by the person with developmental disorder.

Here, the mini-game may be saved to be performed later when the person with developmental disorder wants to perform it later, and the mini-game may have an effect of enhancing the training motivation of the person with developmental disorder and reducing his or her problematic daily behavior.

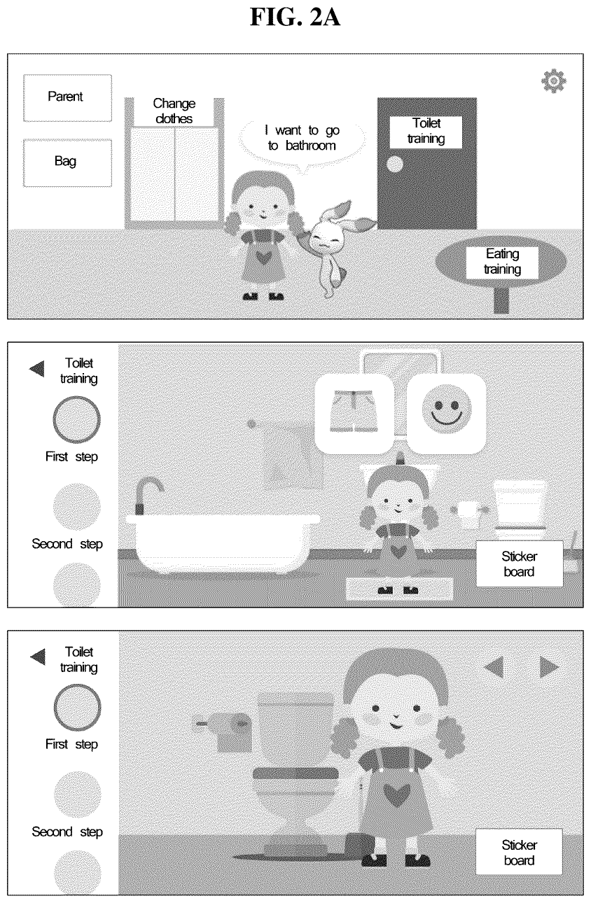

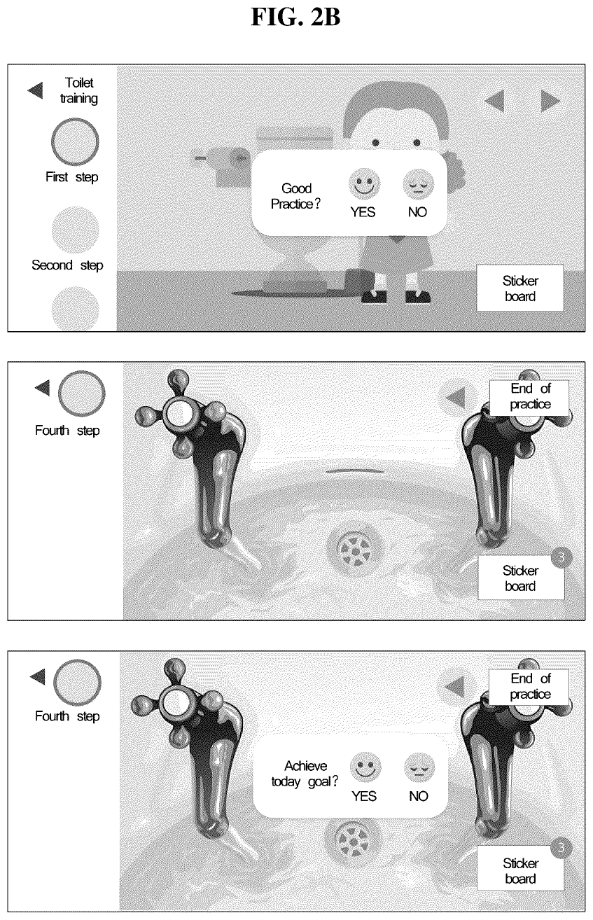

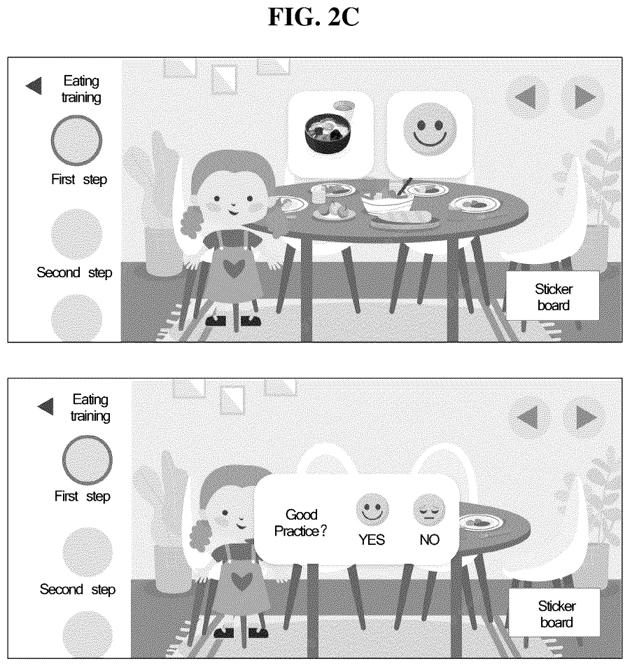

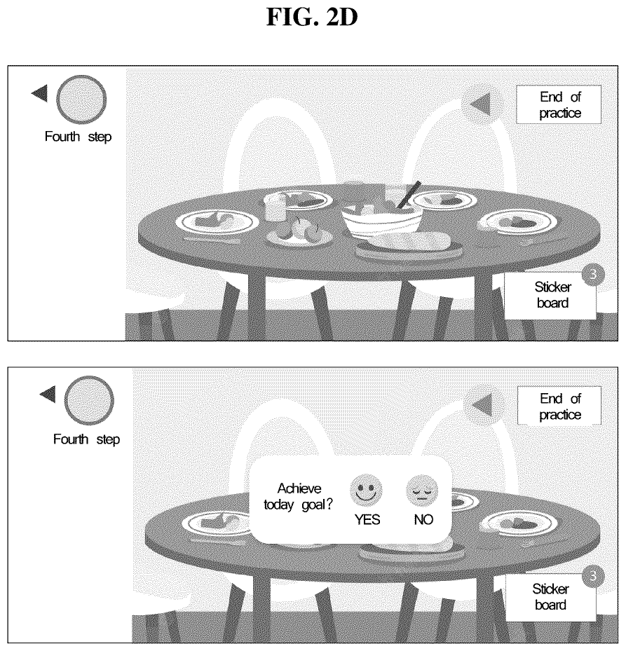

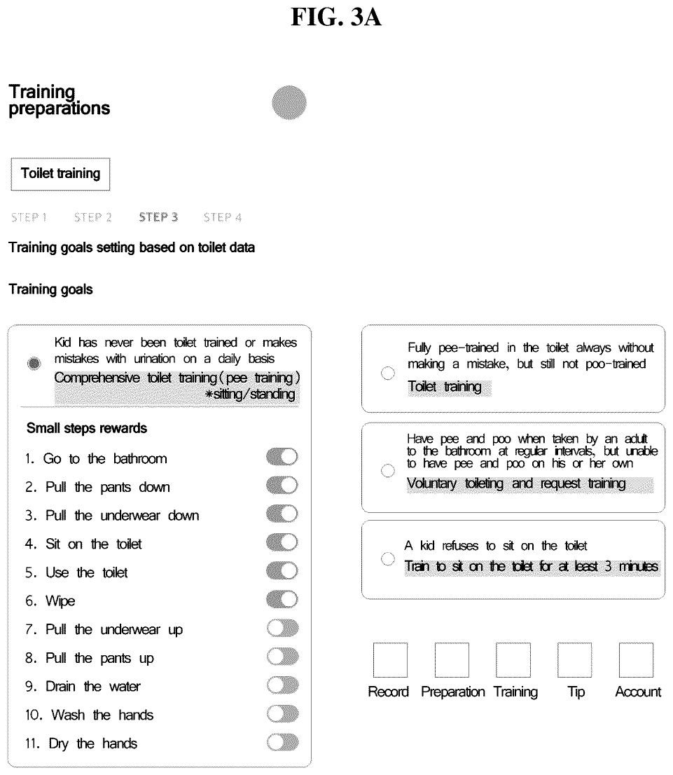

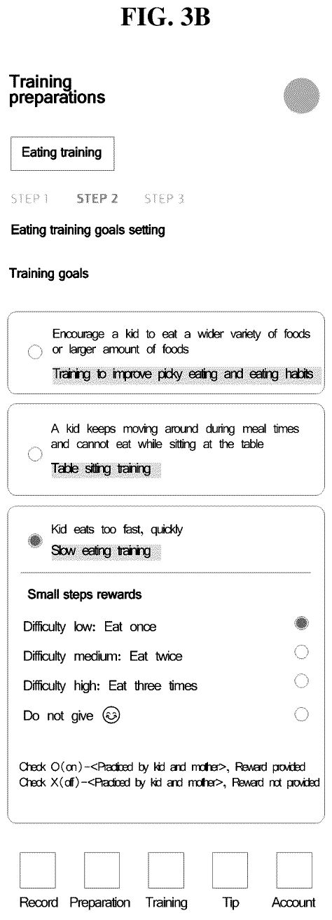

FIGS. 2A-2D are diagrams showing an example of training services provided by a service apparatus according to an embodiment of the present disclosure, and FIGS. 3A and 3B are diagrams showing evaluations of training services in a service apparatus according to an embodiment of the present disclosure.

Referring to FIGS. 2A-2D, 3A and 3B, there is a diagram showing a toilet training and an eating training from among training services provided by the service apparatus 100.

Here, according to a toilet training in FIGS. 2A and 2B and a feeding training in FIGS. 2C and 2D, the guardian of a person with developmental disorder may set a training service based on training service data to perform the training in the training service setting unit 1311 of the guardian mode service unit 131.

Furthermore, the person with developmental disorder may directly perform the toilet training service or eating training service while observing the behavior content of the training character that reflects the set training service.

Accordingly, the guardian may enter behavior information according to the training service, and in response thereto, reward data may be set in the reward system setting unit 1315 as a reward granted to the person with developmental disorder. For example, there may be a sticker, and the shape of the sticker may appear in the shape of an animal or object that can attract the attention of a person with a developmental disorder.

In addition, the guardian may record the behavior data of the person with developmental disorder in the evaluation recording unit 1317 included in the guardian mode service unit 131 while frequently switching between the guardian mode service unit 131 and the person-with-developmental-disorder mode service unit 133, and output result data as a graph on the training result reporting unit 1319.

In this process for the training service, the guardian may select a training service from the guardian mode service unit 131, and set the training goal and training method of the training service based on the training service data.

The training goal of the training service corresponding to the training service data may be customized to the person with developmental disorder to establish a customized training service.

Furthermore, a person with developmental disorder performing the set training service may set a training character that reflects the training service based on the training service data in the training character setting unit 1331 of the person-with-developmental-disorder mode service unit 133 so as to perform the training service, and accordingly, a degree of immersion may be enhanced while the person with developmental disorder performs a training service, and the training service may be more easily performed.

Then, when the training service is terminated, the person with developmental disorder may be provided with a mini-game or a decoration game to be played alone using the data of a final reward.

For a more specific example, as shown in FIG. 3A, when a training service includes a toilet training process, the guardian mode service unit 131 may output a checklist for each step of toilet training of a person with developmental disorder, and output a behavior input interface that allows the guardian's behavior data corresponding to the checklist for each stage to be input.

For example, as a training goal of a toilet training service, a toilet data input may be set in the training service setting unit 1311, and the guardian may enter toilet data as behavior data of a person with developmental disorder entered into the behavior input interface. In addition, the toilet data is recorded in the evaluation recording unit 1317, and the training result reporting unit 1319 may process and output evaluation result data compared to a guideline in a reporting manner.

For example, as shown in FIG. 3A, a user may pre-set training goals for comprehensive toilet training through the training service setting unit 1311, and furthermore, also set detailed training goals corresponding to the training goals.

More specifically, the training goals may include: a first goal ‘Go to the bathroom’, a second goal ‘Pull the pants down’, a third goal ‘Pull the underwear down’, a fourth goal ‘Sit on the toilet’, 5a fifth goal’. ‘Use the toilet’, a sixth goal ‘Wipe’, a seventh goal ‘Pull the underwear up’, an eighth goal ‘Pull the pants up’, a ninth goal ‘Drain the water’, a tenth goal ‘Wash the hands’, and an eleventh goal ‘Dry the hands’, for an example.

Here, the guardian may compare the behavior data of the person with developmental disorder with the first to eleventh goals, check whether the person with developmental disorder has easily performed toilet training, and report and store the check results in real time in the evaluation recording unit 1317 according to an embodiment of the present disclosure, and the result data may be processed through the training result reporting unit 1319.

Furthermore, FIG. 3B relates to an eating training service, wherein the training service setting unit 1311 may pre-set training goals for slow eating training, and furthermore, may also set a detailed training goals corresponding to the training goals.

More specifically, the training goals may be set as a first goal ‘Eat rice slowly once’, a second goal ‘Eat rice slowly twice’, and a third goal ‘Eat rice slowly three times’, and when the person with developmental disorder achieves the first to third goals, a sticker may be provided as pre-set reward data.

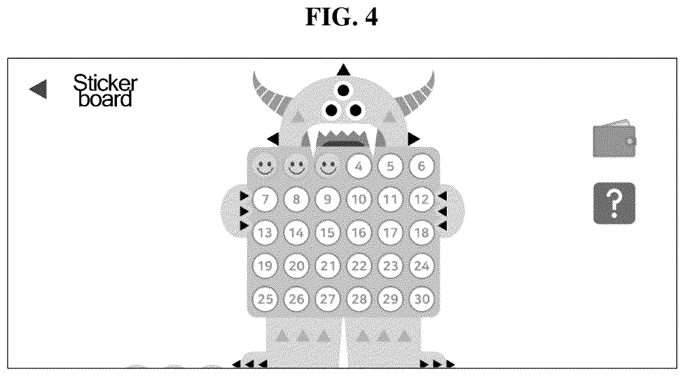

FIG. 4 is a diagram showing an example of a reward interface for training services according to an embodiment of the present disclosure.

Referring to FIG. 4, the reward system setting unit 1315 of the guardian mode service unit 131 included in the service apparatus 100 according to an embodiment of the present disclosure may allow a guardian of a person with developmental disorder to pre-set reward data so as to provide the reward data for training services and game services.

Here, the reward data may include stickers that can motivate the person with developmental disorder in performing the training service, and for an example, a final reward pre-set by the guardian may be granted to the person with developmental disorder when a certain amount of stickers are collected.

Furthermore, when the person with developmental disorder achieves 100% of the collected amount of stickers received as rewards that have been pre-set in the reward system setting unit 1315, the guardian may grant a final reward to the person with developmental disorder through the reward system providing unit 1335, and upload and record a photo thereof.

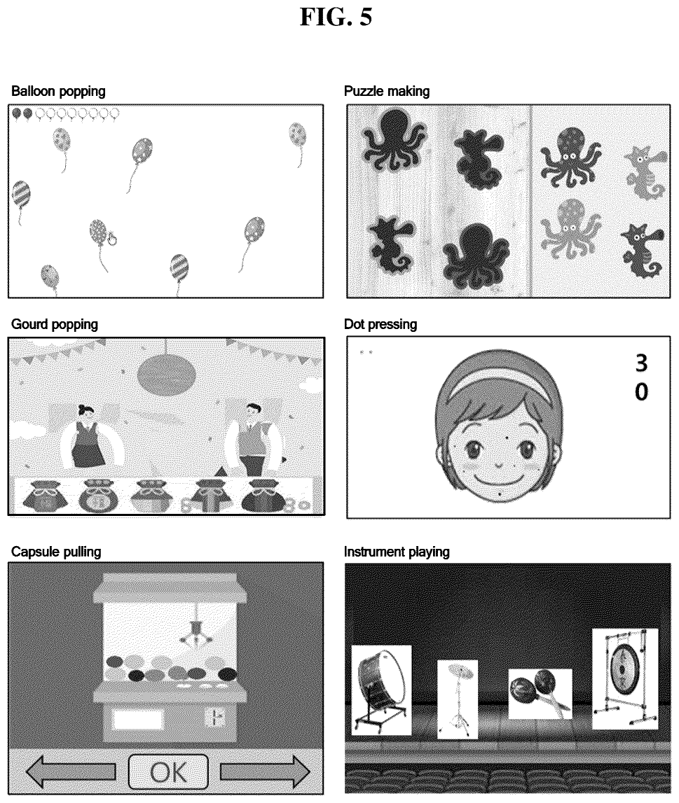

FIG. 5 is a diagram showing game services provided by a service apparatus according to an embodiment of the present disclosure.

Referring to FIG. 5, a game service provided by the service apparatus 100 according to an embodiment of the present disclosure may be set in the game service setting unit 1313.

Here, the game service setting unit 1313 may allow the guardian of the person with developmental disorder to set a game service performed by the person with developmental disorder based on game service data.

In addition, the set game service may be provided from the game service providing unit 1337 of the person-with-developmental-disorder mode service unit 133 when the person with developmental disorder performs a training service.

Here, the game service may be provided in a cooperative game format in which the person with developmental disorder and the guardian play in a cooperative manner, and a mini-game format in which the person with developmental disorder plays alone.

For example, the cooperative game may be a multi-player game in which the person with developmental disorder and the guardian play together to improve a lack of social skills that are the main symptoms of autism spectrum disorder (ASD) and a difficulty in forming bonds with others due to the lack of social skills to enhance sociality and connectedness.

In addition, the mini-game, which is a single-player game given to the person with developmental disorder along with the training service, may be a game played for a short period of time preferred by the person with developmental disorder, thereby having an effect of enhancing the training motivation of the person with developmental disorder as well as improving his or her problematic daily behavior.

For example, the mini-game may be provided to the person with developmental disorder, such as balloon popping, puzzle making, gourd popping, dot pressing, capsule pulling, and instrument playing.

As described above, this mini-game may have an effect of enhancing the training motivation of the person with developmental disorder and improving his or her problematic daily behavior.

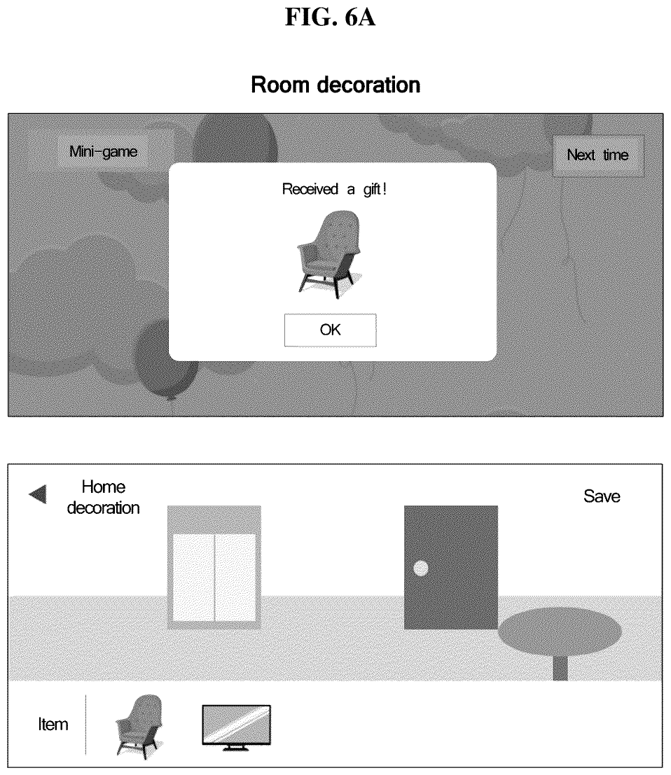

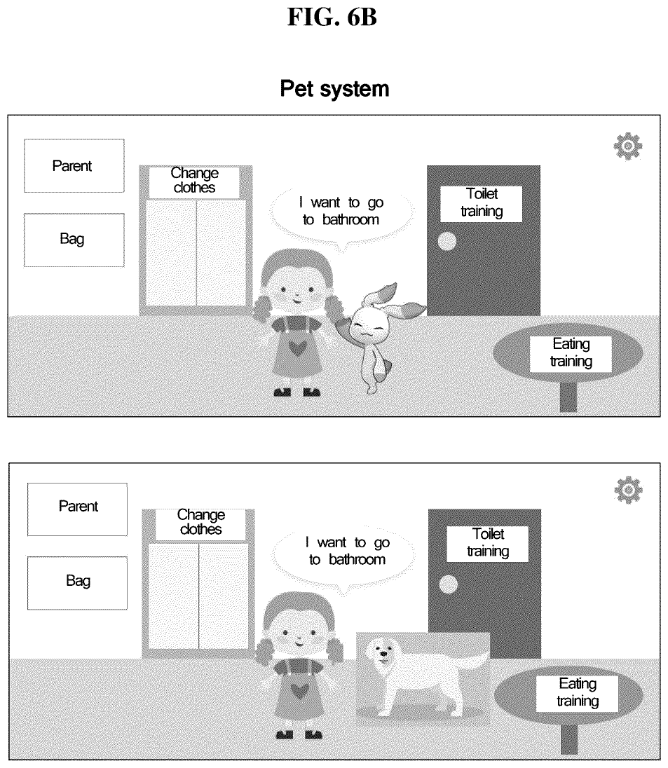

FIGS. 6A and 6B is a diagram showing a reward interface for game services according to an embodiment of the present disclosure.

Referring to FIGS. 6A and 6B, the reward data may be directly set in advance by the guardian of a person with developmental disorder in the reward system setting unit 1315, and may be reward data for a training service performed by the person with developmental disorder, and a mini-game, which is a game service set in the game service setting unit 1313 as the reward data for the training service, may be provided to the person with developmental disorder.

As shown in FIGS. 6A and 6B, the person with developmental disorder may receive furniture items or pets capable of decorating his or her own room through the service apparatus 100 according to an embodiment of the present disclosure, as reward data corresponding to mini-games such as balloon popping, puzzle making, gourd popping, dot pressing, capsule pulling, and instrument playing.

The reward data may enhance the long-term training motivation of the person with developmental disorder, and may be effective in improving his or her daily behavior.

Here, there is an applied behavior analysis (ABA) treatment, which is known to be highly effective in improving the symptoms of the person with developmental disorder. The ABA treatment has a treatment principle in which when the guardian of the person with developmental disorder gives an instruction of ‘A’ as a prerequisite stimulus, in case the person with developmental disorder shows a correct response of ‘B’, praise or reward data of ‘C’ is provided as a result induction or follow-up stimulus so as to reinforce the behavior data of the person with developmental disorder as correct daily behavior.

The main difference of the ABA treatment, unlike general education, is ‘what kind of follow-up stimulation is provided to the person with developmental disorder.’ Anyone may most likely react greatly to reward data received when he or she performs an action worthy of praise, but when he or she does something wrong, discipline is usually given, however, the person with developmental disorder may have a negative impact due to the emotional damage he or she receives from punishment, and thus discipline may be preferably avoided for the person with developmental disorder.

Therefore, as shown in FIGS. 6A and 6B, the reward system providing unit 1335 may allow the person with developmental disorder to receive furniture items or pets to decorate his or her room with reward data for the mini-game, thereby resulting in a reward suitable for the above-mentioned ABA treatment method.

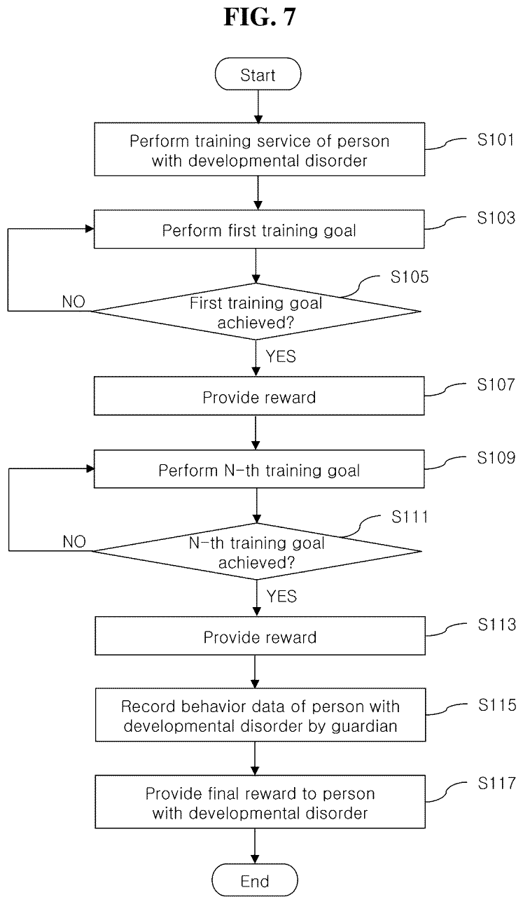

FIG. 7 is a flowchart for explaining a service apparatus according to an embodiment of silver present disclosure.

First, the service apparatus 100 starts performing a training service for a person with a developmental disorder (S101).

Here, the training service setting unit 1311 may set a training service performed by the person with developmental disorder, based on training service data, by the guardian of the person with developmental disorder.

The training service data may include, for example, preparations required for the training service selected by the guardian, an ongoing progress and method of the training service, precautions for performing the training service, a method of creating a training environment, and training goals, and may further include an evaluation guideline for the training service.

Furthermore, the training service data may be set differently depending on the type of training service, but an evaluation step of the training service may be preferably divided into several items according to the training service data to be set as training goals in order to maximize the immersion and training effect for children suffering from major developmental disorders including autism spectrum disorders (ASDs).

Next, the person with developmental disorder performs a first training goal (S103).

Here, the person with developmental disorder may receive the training service reflecting the training character to perform the training service while being accompanied by the guardian.

Accordingly, as described above, the person with developmental disorder may perform training goals set by dividing the training service into several items according to the training service data.

Then, it is determined whether the person with developmental disorder has achieved the first training goal of the training service (S105).

Here, the guardian of the person with developmental disorder may determine whether the first training goal has been achieved by observing the behavior data of the person with developmental disorder.

In addition, when the person with developmental disorder achieves the first training goal, reward data pre-set by the guardian is provided to the person with developmental disorder (S107).

Here, the reward data may be pre-set in the reward system setting unit 1315 to allow the guardian to provide the reward data for the training service to the person with developmental disorder.

Here, when the person with developmental disorder does not achieve the first training goal while performing the training service, the first training goal may be performed again by moving from step S107 to step S103.

Furthermore, when achieving the set training goal, the person with developmental disorder may perform a second training goal, which is a next step, and accordingly, the person with developmental disorder performs an N-th training goal (S109).

Next, it is determined whether the person with developmental disorder has achieved the N-th training goal of the training service (S111).

Here, the guardian of the person with developmental disorder may determine whether the first training goal has been achieved by observing the behavior data of the person with developmental disorder.

In addition, when the person with developmental disorder achieves the first training goal, reward data pre-set by the guardian is provided to the person with developmental disorder (S113).

Here, when the person with developmental disorder does not achieve the N-th training goal while performing the training service, the N-th training goal may be performed again by moving from step S113 to step S109.

As such, the person with developmental disorder may use the service apparatus 100 to perform the training service to improve daily behavior through a training service performance process.

In addition, the guardian records the behavior data of the person with developmental disorder (115).

Here, the guardian may record behavior data of the person with developmental disorder through the evaluation recording unit 1317.

Accordingly, the evaluation recording unit 1317 may allow the guardian to evaluate the training service performed by the person with developmental disorder and record the collected behavior data, and the training result reporting unit 1319 may calculate and output evaluation data formed based on the behavior data recorded in the evaluation record unit 1317 as result data of the training service performed by the person with developmental disorder.

Furthermore, when the person with developmental disorder achieves the N-th training goal, the training service is terminated, and a pre-set final reward is provided to the person with developmental disorder (S117).

Here, when the person with developmental disorder performs the training service, the sticker that can serve as motivation may be received as a reward, and when a certain amount of stickers are collected, a final reward pre-set by the guardian may be granted to the person with developmental disorder, and a photo of the final reward may be uploaded and recorded.

The foregoing method according to the present disclosure may be produced as a program to be executed on a computer and stored in a computer-readable recording medium, and examples of computer-readable recording media include a ROM, a RAM, a CD-ROM, a magnetic tape, a floppy disk, an optical data storage device, and the like.

The computer-readable recording medium may be distributed over computer systems connected via a network, and stored and executed as computer-readable codes in a distributed manner. Furthermore, functional programs, codes, and code segments for implementing the method may be easily inferred by programmers in the technical field to which the present disclosure pertains.

While the preferred embodiments of the present disclosure have been shown and described above, it will be of course understood by those skilled in the art that various modifications may be made without departing from the gist of the disclosure as defined in the following claims, and it is to be noted that those modifications should not be understood individually from the technical concept and prospect of the present disclosure.

What is claimed is:

1. A service apparatus comprising:
a user account management unit that manages an account of a person with developmental disorder and a guardian of the person with developmental disorder;
a training service information output unit that outputs training service information for improving the daily behavior of the person with developmental disorder; and
a mode switching service unit that processes mode switching between a guardian mode and a person-with-developmental-disorder mode to output the training service information.

2. The service apparatus of claim 1, wherein the mode switching service unit comprises:
a guardian mode service unit that sets service data corresponding to the training service information in the guardian mode;
a person-with-developmental-disorder mode service unit that outputs the training service information configured according to the set service data in the person-with-developmental-disorder mode; and
a database unit that stores and updates the service data shared in the guardian mode service unit and the person-with-developmental-disorder mode service unit in real time.

3. The service apparatus of claim 2, wherein the guardian mode service unit comprises:
a training service setting unit that sets a training goal for a training service performed by the person with developmental disorder based on the service data.

4. The service apparatus of claim 2, wherein the guardian mode service unit comprises:
a game service setting unit that sets a game service performed by the person with developmental disorder based on the service data.

5. The service apparatus of claim 2, wherein the guardian mode service unit comprises:
a reward system setting unit that pre-sets, when behavior data of the person with developmental disorder is input from the guardian in response to the output of the training service information, reward data to be provided to the person with developmental disorder based on the behavior data.

6. The service apparatus of claim 2, wherein the guardian mode service unit comprises:
an evaluation recording unit that records, when behavior data of the person with developmental disorder is input from the guardian in response to the output of the training service information, collected behavior data; and
a training result reporting unit that calculates and reports evaluation data based on the behavior data recorded in the evaluation recording unit.

7. The service apparatus of claim 2, wherein the person-with-developmental-disorder mode service unit comprises:
a training character setting unit that sets a training character reflecting the service data; and
a training service providing unit that reflects the set training character in video content of the training service information to induce the person with developmental disorder to follow the training character of the training service information.

8. A method of operating a service apparatus, the method comprising:
a user account management step of managing accounts of a person with developmental disorder and a guardian of the person with developmental disorder;
a training service information output step of outputting training service information for improving the daily behavior of the person with developmental disorder; and
a mode switching service step of processing mode switching between a guardian mode and a person-with-developmental-disorder mode to output the training service information.

9. The method of claim 8, wherein the mode switching service step comprises:
a guardian mode service step of setting service data corresponding to the training service information in the guardian mode;
a person-with-developmental-disorder mode service step of outputting the training service information configured according to the set service data in the person-with-developmental-disorder mode; and
a step of storing and updating the service data shared in the guardian mode service unit and the person-with-developmental-disorder mode service unit in real time.

10. The method of claim 9, wherein the guardian mode service step comprises:
a training service setting step of setting a training goal for a training service, step by step, performed by the person with developmental disorder based on the service data.

11. The method of claim 9, wherein the guardian mode service step comprises:
a game service setting step of setting a game service performed by the person with developmental disorder based on the service data.

12. The method of claim 9, wherein the guardian mode service step comprises:
a reward system setting step of pre-setting, when behavior data of the person with developmental disorder is input from the guardian in response to the output of the training service information, reward data to be provided to the person with developmental disorder based on the behavior data.

13. The method of claim 9, wherein the guardian mode service step comprises:
an evaluation recording step of recording, when behavior data of the person with developmental disorder is input from the guardian in response to the output of the training service information, the collected behavior data; and
a training result reporting step of calculating and reporting evaluation data based on the behavior data recorded in the evaluation recording unit.

14. The method of claim 9, wherein the person-with-developmental-disorder mode service step comprises:
a training character setting step of setting a training character reflecting the service data; and
a training service providing step of reflecting the set training character in video content of the training service information to induce the person with developmental disorder to follow the training character of the training service information.

15. A computer program stored in a computer-readable medium for executing the method according to claim 8 on a computer.
